(19) United States
(12) Patent Application Publication
(10) Pub. No.: US 2002/0169442 A1
(43) Pub. Date: Nov. 14, 2002

(54) Device and a method for treating skin conditions

(76) Inventor: Joseph Neev, Lake Forest, CA (US)

Correspondence Address: Albin H. Gess, Price and Gess, 2100 S.E. Main Street, Suite 250, Irvine, CA, 92614-6238, US

(21) Appl. No.: 10/150,632

(22) Filed: May 17, 2002

Related U.S. Application Data
(63) Continuation-in-part of application No. 09/694,738, filed on Oct. 23, 2000.
Application No. 09/694,738 is a continuation of application No. 09/132,537, filed on Aug. 11, 1998, now Pat. No. 6,168,590.
(60) Provisional application No. 60/055,577, filed on Aug. 12, 1997.

(51) Int. Cl. A61B 18/20

(52) U.S. Cl. 606/9; 606/10

(57) ABSTRACT

Skin tissue is subjected to energy that creates differential heating of the area being treated causing pores and follicle ducts to open so that excess oil, sebum, fatty deposits, or other unwanted deposits can be removed. A vacuum device is used to direct suction to the treated skin area helping to remove the unwanted deposits. The heating is controlled so that no skin tissue is damaged while still providing enough heat to the sebaceous gland to alter the gland to reduce its production of sebum and destroy bacteria in the treated area.

### CROSS-REFERENCE TO RELATED APPLICATIONS

This application is a continuation-in-part of U.S. application Ser. No. 09/694,738 filed on Oct. 23, 2000 for Method and Apparatus For Permanent Hair Removal which is a continuation of U.S. application Ser. No. 09/132,537 filed on Aug. 11, 1998 for a Method For Permanent Hair Removal, now U.S. Pat. No. 6,168,590 granted Jan. 2, 2001 which claims the benefit of the filing date of U.S. Provisional Application Serial No. 60/055,577, filed on Aug. 12, 1997 for Method and Apparatus For Selective Hair Removal and Modification.

### FIELD OF THE INVENTION

The present invention relates generally to the application of energy to biological tissue, and specifically to the application of electromagnetic energy to the skin in order to treat various skin diseases.

### BACKGROUND OF THE INVENTION

It is known in the art to apply electromagnetic energy to biological tissue to engender changes therein. Sunbathers, for example, regularly expose themselves to bright sunlight in order to increase melanocyte activity in the basal layer of the epidermis, responsive to the sun's ultraviolet (UV) radiation. Artificial UV sources have been created to satisfy the desire for a “healthy”-looking tan in the winter. Other forms of electromagnetic energy, laser-light in particular, are currently used in a large range of therapeutic and cosmetic procedures, including eye surgery, hair removal, wrinkle removal, and tattoo removal.

PCT publication WO 98/55035, which is incorporated herein by reference, describes methods for minimizing injury to biological tissue surrounding a site exposed to pulses of electromagnetic energy.

U.S. Pat. No. 5,720,894 to Neev et al., which is incorporated herein by reference, describes biological tissue processing using Ultrashort Pulse High Repetition Rate Laser System for Biological Tissue Processing.

It is known in the art to use UV and blue light to cure acne. A beam of short wavelength light is irradiated and is supposed to destroy bacteria through sterilizing ability of the high energy photon to disrupt molecular bond and photochemical destruction of living cells. This method is deficient however, because of the relatively short depth of penetration of the short wavelengths regime and the danger of mutagenetic effect as well as the effective shielding of deeper lying bacteria by superficial skin structures.

It is also known in the art to use chemical peels and Retin A to chemically peel of the outer layer of the skin. This method is deficient however, because of side effect, long response time and longer time duration between application of the treatment and results and various side effects.

It is also known in the art to apply antibiotic to patients in order to combat active acne. This method is deficient however, since the application of antibiotic is non-selective, often done systemically and thus effect the entire body, and also for the fact that various organisms and bacteria develop resistance to antibiotics and in fact, expose the entire body to increase danger in exposure to bacteria that is now resistance to antibiotics.

It is also known in the art to combat active acne by treating and controlling hormonal activity within a patient body. Again, this is a systemic approach that suffers from many side effects including, in some cases, severe depression, and impact on the entire body.

It is therefore, desirable, to have a simple, non-invasive, non-systemic treatment method and apparatus for the treatment and cure of acne, that, when applied, are free of side effect, yet safe and effective. It is also desirable to have a method that is easy to apply and is relatively quick and easy to administer and produce rapid skin response, relief of symptoms, and cure for the condition.

It is therefore, desirable, to have a simple, non-invasive, non-systemic treatment method and apparatus for the treatment and cure of other skin diseases and skin conditions, that, when applied, are free of side effect, yet safe and effective.

### OBJECTS AND SUMMARY OF THE INVENTION

It is an object of some aspects of the present invention to provide improved apparatus and methods for applying energy to a material.

It is another object of some aspects of the present invention to provide improved apparatus and methods for removing heat generated during application of electromagnetic energy to a material.

It is a further object of some aspects of the present invention to provide improved apparatus and methods for removing heat generated during application of electromagnetic energy to biological tissue.

It is still a further object of some aspects of the present invention to provide improved apparatus and methods for decreasing pain during application of electromagnetic energy to biological tissue.

It is yet a further object of some aspects of the present invention to provide improved apparatus and methods for performing medical treatments.

It is also an object of some aspects of the present invention to provide improved apparatus and methods for performing cosmetic treatments.

It is also an object of some aspects of the present invention to provide improved apparatus and methods for healing of skin diseases and skin illnesses.

It is further an object of some aspects of the present invention to provide improved apparatus and methods for enabling electromagnetic energy source to allow healing of skin diseases and skin illnesses or improved conditions.

It is yet a further object of some aspects of the present invention to provide methods and apparatus for enabling a chemical, RF, Microwave, mechanical, electric, magnetic, or ultrasound energy to advance healing skin diseases and skin illnesses.

It is also an object of some aspects of the present invention to provide improved methods and apparatus for enabling a low-power electromagnetic energy source to advance healing skin diseases and skin illnesses substantially without pain, while substantially minimizing the amount of damage or modification to remaining tissue.

It is also an object of some aspects of the present invention to provide improved methods and apparatus for enabling a low-power electromagnetic energy source to perform skin treatment, treatment of acne and treatment that prevent the occurrence of acne.

It is also an object of some aspects of the present invention to provide improved methods and apparatus for enabling a low-power electromagnetic energy source to perform tissue treatment that cures acne and relieves symptoms of acne.

In a preferred embodiment of the present invention, the tissue of the skin is subjected to localized heating that for a given time and in a defined location, elevates the temperature of the skin in one location as compared to an its normal temperature. This elevation of skin temperature corresponds to expansion and displacement of a portion of the skin thus leading to opening of skin pores.

In another preferred embodiment of the present invention, the tissue of the skin is subjected to localized heating that for a given time and in a defined location, elevates the temperature of the skin in one location as compared to an adjacent location. This elevation of skin temperature corresponds to expansion and displacement of a portion of the skin with respect to an adjacent location thus leading to opening of skin pores.

In a further preferred embodiment, an intermediate substance which is capable of absorbing at least a portion of the electromagnetic energy from a source, is placed between the energy source and the skin, absorbs the source energy and converts it to heat. Being in contact with the skin, the substance elevates the temperature of the skin to cause to an expansion and displacement leading to opening of skin pores and relieving of acne conditions.

In yet a further preferred embodiment, an intermediate substance which is capable of absorbing at least a portion of the electromagnetic energy from a source, is placed between the energy source and the skin, absorbs the source energy and converts it to heat. Being in contact with the skin, the substance elevates the temperature of the skin in one location as compared to an adjacent location. This elevation of skin temperature corresponds to expansion and displacement of a portion of the skin with respect to an adjacent location thus leading to opening of skin pores.

### BRIEF DESCRIPTION OF THE DRAWINGS

FIG. 1 is a simplified diagram of an apparatus for treating skin in order to prevent and cure acne and other skin conditions in accordance with a preferred embodiment of the present invention;

FIG. 2 is a simplified diagram of an apparatus for treating skin in order to prevent and cure acne and other skin conditions in accordance with a preferred embodiment of the present invention;

FIG. 3 is a simplified diagram of an apparatus for treating skin in order to prevent and cure acne and other skin conditions in accordance with a preferred embodiment of the present invention;

FIG. 4 is a simplified diagram of a method for treating skin in order to prevent and cure acne and other skin conditions in accordance with a preferred embodiment of the present invention;

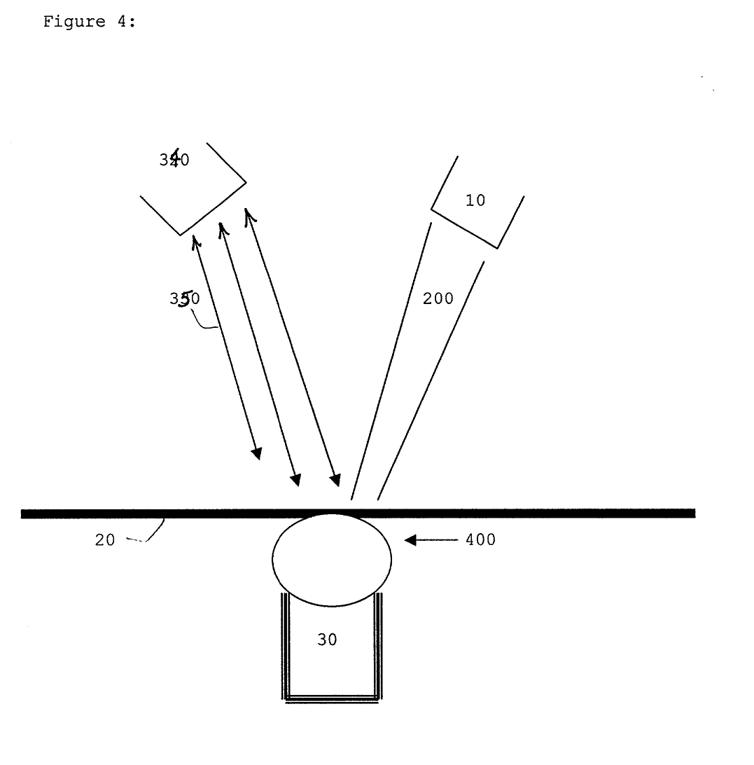

FIG. 5 is a graph of a temperature profile generated for treating skin in order to prevent and cure acne and other skin conditions in accordance with a preferred embodiment of the present invention;

FIG. 6 is a schematic illustration of a preferred embodiment illustrating a device for treating skin according to the present invention; and

FIG. 7 is a diagrametic illustration of a cross-section of skin temperature changes that occur in sections of the skin as the result of the present invention.

### DETAILED DESCRIPTION OF PREFERRED EMBODIMENTS

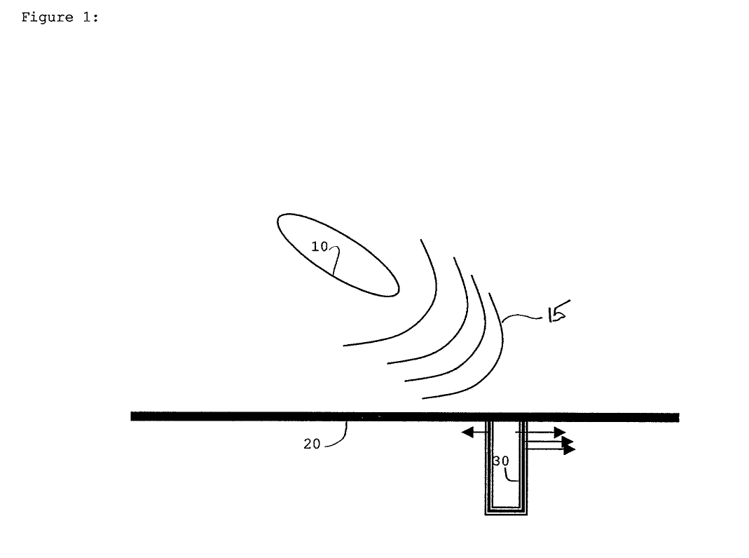

Referring first to FIG. 1 which illustrates a preferred embodiment that contemplates a device comprising an energy source 10, capable of displacing a portion of the skin 20, so that at least one skin pore 30 is forced to open so that excess oil, sebum, fatty deposits, or other unwanted deposits can be removed from the pore.

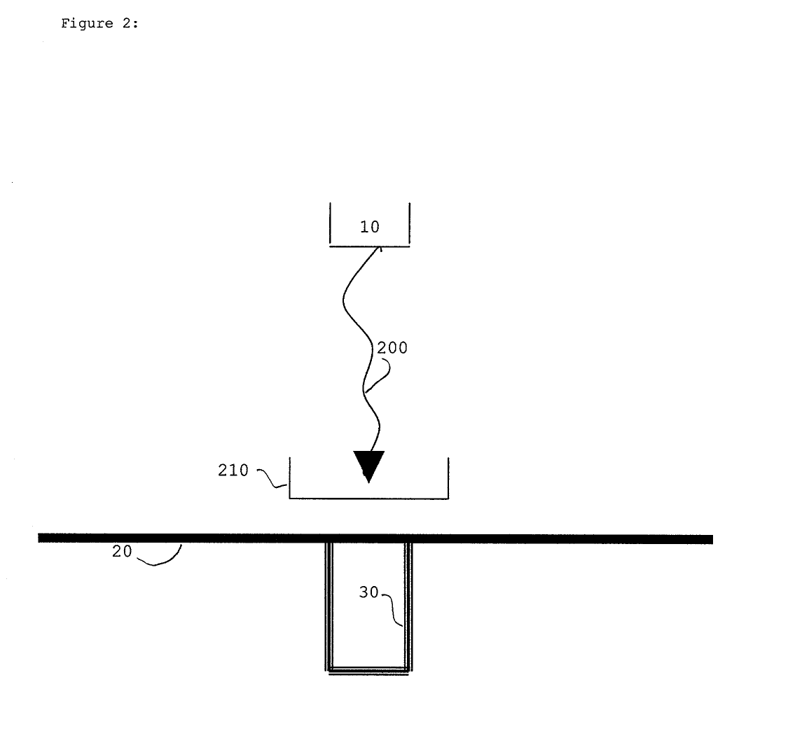

FIG. 2 illustrates another simplified diagram for treating acne and other skin conditions in accordance with a preferred embodiment of the present invention. Here, the energy source 10 is a source that emits a beam of electromagnetic energy 200 carrying particles (photons). The beam 200 of photons is then intercepted and absorbed by an intermediate material 210 which contains a substance capable of absorbing photons. The energy of the photons absorbed by the substance 210 is converted to heat which, in turn, is conducted to the skin 20. The thermal energy thus deposited on the skin forces at least one skin pore 30 to open so that excess oil, sebum, fatty deposits, or other unwanted deposits can be removed from the pore.

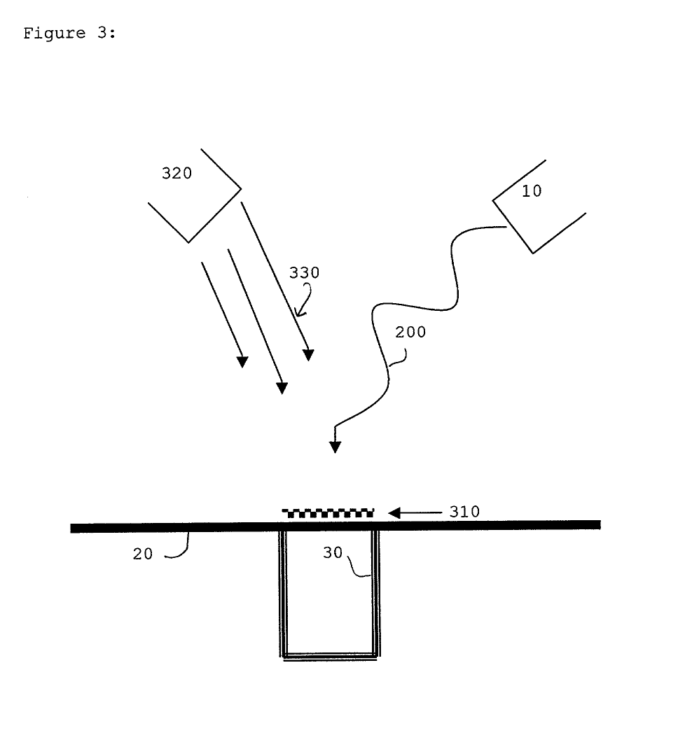

FIG. 3 illustrates another simplified diagram for treating acne. Here, the energy source 10 is a source that emits a beam of electromagnetic energy 200 carrying particles (photons). A substance 310 capable of absorbing the photons is applied to the skin. The energy of the photons impinging on the substance 310 is converted to heat energy which, in turn, is either partly conducted to the skin 20, or results in ablative or evaporative explosive removal of the top layer of the skin. The thermal energy thus deposited on the skin forces at least one skin pore 30 to open so that excess oil, sebum, fatty deposits, or other unwanted deposits can be removed from the pore. If evaporation or ablation is achieved, opening of the pores may be accomplished, at least in part, by removal of material that is blocking the pore or hair duct openings at the surface of the skin. In a variation of this preferred embodiment, the substance 310, capable of absorbing the photons is substantially removed from most of the skin prior to irradiation by the photon beam 200, except that some of the high absorption substance 310 is left in and around the location of the pore or hair duct opening.

A variation of this preferred embodiment includes a heat removal phase that causes rapid contraction. This phase follows the photon and heat phase. A source of coolant material 320 is caused to direct a coolant substance 330 to the area of the skin being treated.

Another embodiment contemplates an energy source 10 which emits a beam of electromagnetic energy 200 carrying particles, i.e., photons. The beam of photons is focused into a spatial location 400 underneath the skin surface to achieve subsurface heating of layers inside the skin 20. The energy of the photon is converted to heat energy in the skin 20. The thermal energy in the skin forces at least one skin pore 30 to open so that excess oil, sebum, fatty deposits, or other unwanted deposition can be removed from the pore.

A source of vacuum 340 may be used after this step. Vacuum source 340 will be activated following the heating and displacement phases to enhance the removal of unwanted substance from the skin. By applying a vacuum suction action 350 to the surface of the skin, the unwanted substance in the skin pore 30 or hair duct is removed.

A variation of this preferred embodiment utilizes a heat removal and rapid contraction phase following the photon heat depositions when a substance capable of removing the energy from the skin is applied to the skin surface.

In its most general form, the device contemplated by the present invention contains an energy source capable of modulating physical conditions of the skin by heating or mechanically modulating a portion of the skin to treat skin diseases and skin conditions. This embodiment may also include components to allow focusing the energy from the energy source 10 so that a portion of the skin 20 is displaced with respect to an adjacent portion of the skin. Such relative differential displacement may allow loosening of skin components that block efficient drainage of the pores and hair follicle openings to relieve acne and other undesirable skin conditions.

The device contemplated by the present invention may contain an energy source capable of modulating skin physical conditions by heating wherein the heat is also capable of sterilizing an infected area underneath the skin surface thus improving skin conditions such as acne and curing skin diseases. In a further improvement, the heat is also capable of reaching deeper into the skin and changing the condition of the sebaceous gland so that they produce less sebum, or even no sebum, which is a long term advantage in eliminating acne problems.

The energy source utilized may include, but is not limited to light energy, electromagnetic energy, laser energy, RF energy, microwave energy, ultrasound energy, mechanical energy, or chemical energy. Electromagnetic energy, RF energy, light energy, and microwave energy when absorbed in the skin tissue results in expansion and displacement of one portion of the skin with respect to the other. Temperature increases in the range of 15° C. to 250° C. achieve optimal results both in terms of displacement as well as in terms of sterilization impact on the uppermost layers of the skin.

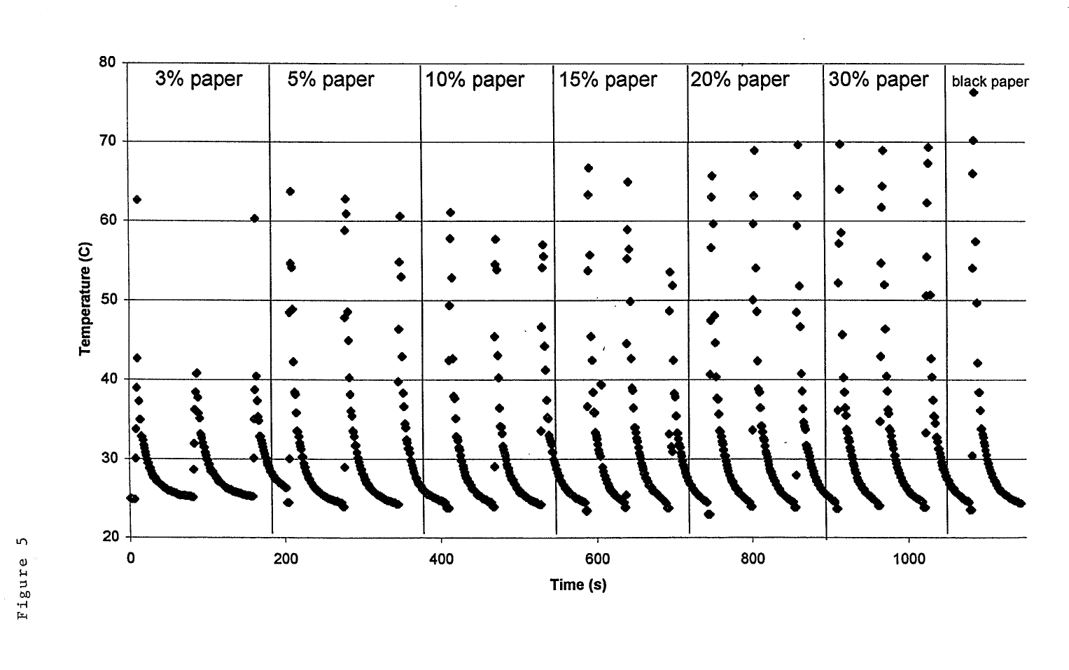

FIG. 5 shows the temperature generated with a device constructed in accordance with the present invention. It shows that temperatures as high as 70° C. and 80° C. can be generated. Physical Properties of Tissue by F. A. Duck shows that the thermal expansion coefficient of human fat is 19.9×10⁴/0° C. For a volume of a 100 micrometer cube, an increase in temperature on the order of from about 50° C. to about 100° C. will result in a thermal expansion ratio on the order of from about 20 micrometers to about 30 micrometers. Thus, if the skin undergoes a temperature increase of 50° C. to a depth of about 100 micrometers, one can expect a sebum driven expansion of about 20 micrometers. This is about one fourth the size of an average human hair follicle.

Besides utilizing an energy source capable of modulating the physical conditions of the skin, the present invention contemplates using a vacuum source for applying suction to the surface of the skin following the energy deposition to enhance removal of unwanted substances from the skin.

An energy source that delivers mechanical energy directly to the skin to effect displacement of the surface of the skin to allow opening and cleaning of skin pores and follicle openings may be used instead of thermal generating energy. Ultrasound energy generates thermal energy in the skin causing displacement in the skin and opening and cleaning the skin pores and follicle openings.

If thermal energy is being applied to the skin, an intermediate element capable of conducting the thermal energy to the skin sufficient to open and clean skin pores and follicle openings is desirable. If electromagnetic energy is being used, the intermediate element should contain, at least in some portion, a substance that allows absorption of electromagnetic energy and converts it into heat.

If the electromagnetic energy being used is in a spectral range that is absorbed well by the skin, localized heating of the skin and differential displacement of some of the skin volume can be accomplished by simply focusing the beam of electromagnetic energy directly on the skin.

If the source of energy is laser energy, energy in the visible range of the spectrum can be used as generated, for example, from a solid state diode laser. The energy from the laser is directed to an intermediate substance capable of both absorbing the laser energy and transmitting the absorbed laser energy to the skin. The intermediate substance can be deposited on an intermediate medium, such as a film made of polymers or agar containing a substance that absorbs the laser energy. Alternatively, a thin paper medium containing a substance that absorbs the laser energy may be used.

The intermediate medium is brought into contact with the skin and the laser is activated. The laser beam may be focused to a small spot to maximize power density. Spot size diameter can range from about 1 micrometer to about 10 mm and preferably is from about 100 micrometer to about 2 mm. A Continuous Wave (CW) solid state laser may be used as an energy source. The power may vary from about 1 mw to about 200 W, and preferably the range should be from about 0.2 W to 15 W. The tight focused spot may be moved around a targeted skin surface area using scanners so that a transient energy deposition occurs in each local spot with dwell time ranging from about 1 microsecond to as long as about 10 seconds, and preferably from about 1 ms to about 50 ms.

FIG. 5 shows the range of temperatures that can be generated with a device contemplated by the present invention. For example, a CW solid state laser system of 1 W output power and 808 nm wavelength with a spot size of 200 micrometers in diameter and scanning over an area of 1 cm by 1 cm in about 2 seconds generates a fluence of about 2 Joule per square cm, with dwell time of about 1 ms. The temperatures that can be generated are on the order of 30° C. to 90° C., depending on the density of the laser energy absorbed by the absorbing material. The density of the absorbers may range form about 3% coverage of the intermediate material surface to as mach as about 100% coverage of the intermediate material, and preferably from about 30% to about 100% coverage of the intermediate material surface, with the substance being capable of high absorbence of the laser energy.

A vacuum source is preferably also used to suction the smoke generated during the interaction, as well as to remove debris and unwanted material from the treated skin surface, the hair follicle opening, and skin pores.

The intermediate absorbing material may contain a laser absorbing substance arranged in patterns that maximize the penetration of light to depths of between about 100 micrometers to about 1 mm in order to maximize penetration of the light to heat the sebaceous glands and minimize secretion of the sebum. The preferred density for deeper laser light penetration and direct light heating of the upper layers of the skin is between about 0% to about 70% and preferably (if surface heating utilizing the intermediate absorbing material is taken into account) between 3% and 50%. A laser in the blue to ultraviolet range may be used in order to utilize the sterilization effect of these wavelengths, as well as generating heat. The heat generation will be increased due to the increased absorption resulting from the shorter wavelengths. In addition, blue and green to orange wavelengths are more readily absorbed in the hemoglobin in the blood and thus enhance heat generation at the surface of the skin and in layers below the surface.

It is preferable to use a focusing element to focus the energy from the energy source on a smaller region of the skin. For example, a focused spot may be as small as about 1 micrometer or as large as about 10 mm and preferably between about 10 micrometers and about 2 mm. Such a spot size will create localized heating that results in differential temperature between one spot on the skin surface and an adjacent spot. The expansion of the heated spot will force displacement and opening of the pores and hair follicle openings. The heating of the skin will also result in sterilization of bacteria and lowering the ability of the sebaceous gland to produce sebum. A scanner or other similar element may be use to move the focused beam across a larger treatment area (for example, a total treatment area from about 2 mm by 2 mm to about 10 cm by 10 cm and preferably from 4 mm by 4 mm to about 5 cm by 5 cm). Treatment time for this area should be from about 0.2 second to about 10 seconds, and preferably from about 0.5 second to about 5 seconds.

A willfully triggered substance capable of removing thermal energy (cool air, coolant spray, cryogen spray, liquid nitrogen spray, or a jet Freon-like substance, for example) from the skin at a desired time is also desirable. The activation of the cooling substance to remove energy from the skin surface or the intermediate material surface can be applied before, during, or after the application of energy to the skin surface, but preferably is applied only during or after the application of energy to the surface.

If the energy source used is microwave energy, it is applied to the surface of the skin to allow generation of heat and localized displacements in accordance with principles of the present invention.

If the energy source used is electric energy, it is applied to the surface of the skin to allow generation of heat and localized displacements in accordance with the principle of the present invention. DC or alternating currents, for example, may be applied to the skin tissue and will result in localized heating in accordance with the principles of the present invention.

If the energy source used by the device is plasma energy, the electrically charged electrons and ion gas that is the plasma carries significant energy that can be applied to the surface of the skin. This energy allows generation of heat and localized displacement as well as surface cell removal, and removal of unwanted deposits form the surface of the skin in accordance with principles of the present invention.

If the energy source used by the device is chemical energy, the chemical energy can be applied to the surface of the skin to cause material removal and removal of unwanted deposits from the surface of the skin further allowing opening of skin pores and hair duct openings, in accordance with principles of the present invention. The chemical energy may also generate localized displacements in accordance with principles of the present invention.

If the energy source used by the device is ultrasound energy, the ultrasound energy can be applied to the surface of the skin to cause surface spatial displacement as well as generation of heat in accordance with principles of the present invention.

Forms of mechanical energy may also be applied to the skin to cause localized displacement of portions of the skin, thus leading to unplugging and opening of skin pores and hair follicles.

In a preferred embodiment, the method of the present invention contemplates applying energy to the outer layer of the skin so that the temperature of the top layer of the skin, for example, the top 10 to 50 micrometers of the epidermis are raised to a temperature of between 100° C. to 200° C. above the normal skin temperature. Energy flux of 1 Joule/cm² would raise the temperature of a water-like substance (for example body cells) to a temperature of about 250° C., if heated to a depth of 10 micrometers. A laser source of 1 W scanned at a rate of 1 cm² per second will dwell on a beam spot of about 100 micrometers in diameter for about 0.1 ms. If the energy in our example is deposited at the surface of the skin, in this time duration, the heat will diffuse down to a depth of about 10 micrometers and with water-like material heat capacity of about 4.2 Joules/gram/°C., will cause the temperature rise to be about 250° C.

One important element that is exploited by the present invention is value of the thermal coefficient expansion of fatty material which is about 200,000 per degree C for fat, but only about one tenth of this value in water or cells. Thus, in the top ten microns or so, the temperature rise is going to create an expansion ratio of about 30% to 40% in fat but only 4% in water. If a follicle opening about 100 micrometers in diameter is substantially filled with sebum, the sebum will expand to 30% over its original volume. Sufficient pressure is thus generated for opening of the follicles and skin pores.

A 10 ms heat application will diffuse down to about 100 micrometers in a water-like material. For the same amount of energy input as described above (1 Joule/cm²) the average temperature rise is only 25° C. However, even this temperature rise will result in about 4% volumetric thermal expansion in fatty tissue. Thus, significant expansion can be achieved even at these low levels.

If the temperature rise of 100° C. to 200° C. occurs for a longer time duration or at a higher energy level, fusion of the collagen around the follicle could occur and worsen the blocking of the sebaceous gland drainage, actually aggravating the problem.

For example, after 10 ms, heat deposited at the skin surface, will diffuse in water like tissue, to a depth of about 100 micrometers. The relationship DT=DE/C where C is the material specific heat in Joules/gram/° C., DT is the temperature rise in °C., and DE is the input energy to the volume being heated in Joules, is used. To achieve 100° C. throughout the exemplary follicle volume of 100 micrometer diameter to 100 micrometer depth, 4 Joules of energy are needed. Similarly, to achieve DT=60° C. about the thermal denaturation temperature of tissue, about 2 Joules of energy are needed. Such temperature rises in the lower epidermis or upper dermis may result in both patient discomfort and in clogging or even fusing of the collagen around the follicle creating permanent or longer lasting blockage of the follicle ducts and skin pores. Such a temperature impact may be beneficial however, by damaging the sebaceous glands thus reducing sebum production, but will not be helpful in treating immediate inflamed, or active acne.

An example of parameters used in a device constructed according to the principle of the present invention are shown in the table below.

| 0.6 W = input power | Position | Time (Sec) | Fluence (J/cm²) | |
|---|---|---|---|---|
| | 3 | 3.5-12 | 2.1 | Sebaceous gland modification |
| | 2 | 2.5 | 1.5 | Used effectively |
| | 1 | 1.5 | 0.9 | Used Effectively |

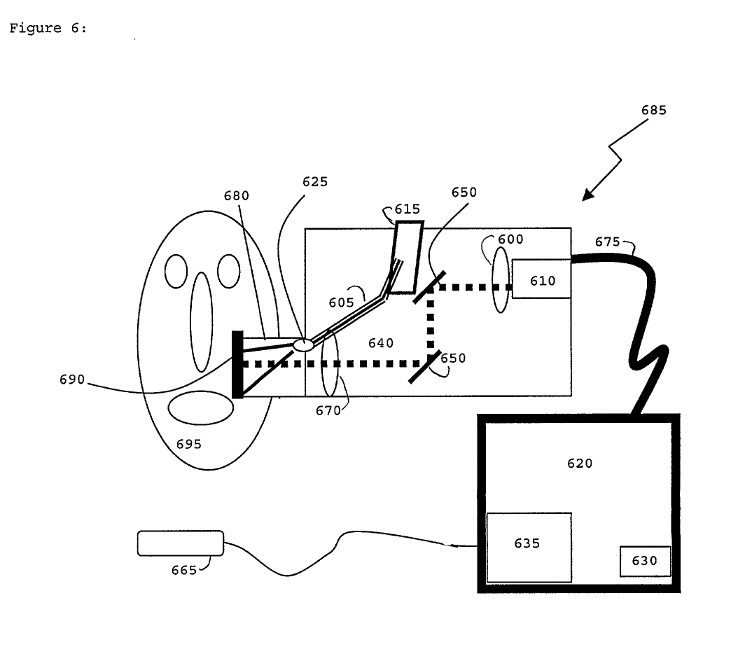

A preferred embodiment of a device 685 for treating skin conditions is illustrated in FIG. 6.

An energy source 610 is triggered by a signal from a control box 620 which contains a microprocessor 630. The activation of the energy source sends a beam through a collimator 600 to two scanner mirrors 650 and a focusing lens 670. The beam then impinges onto an intermediate material (for example the Opto-thermal converter) attachment 680 and a high absorbing substance film 690. The Opto-thermal converter attachment and the high absorbing substance film are in physical contact with the patient's skin 695.

A typical energy source 610 would be a 1 W to 4 W infrared laser, for example, at 810 nm, with a spot size of between 2 micrometers and 2 millimeters. The beam is scanned with a dwell time of 0.1 ms and up to about 0.5 second to cover an area of about 10 mm×10 mm.

The device 685 may also contain a source for a substance 615 capable of removing energy from the intermediate material 680. The flow of the energy removal substance is directed towards the intermediate high absorbing substance film 690 by a tube 605. The flow amount is controlled by an electronic valve 625 which is controlled by the microprocessor 630 in the control box 620. The microprocessor synchronizes the triggering of the energy source 610, the substance capable of energy removal 615, and the two scanners 650 for moving the energy beam around.

The control box 620 may further include a vacuum pump 635 capable of keeping the treatment area clean through an evacuation tube 655. Alternatively, the vacuum pump 635 can be used to create vacuum suction at the skin for cleaning the skin surface, pores and follicle openings. This action is accomplished through the vacuum contact head 655.

The operation control commands from the control box 620 to the device 685 is through an electronic cable 675.

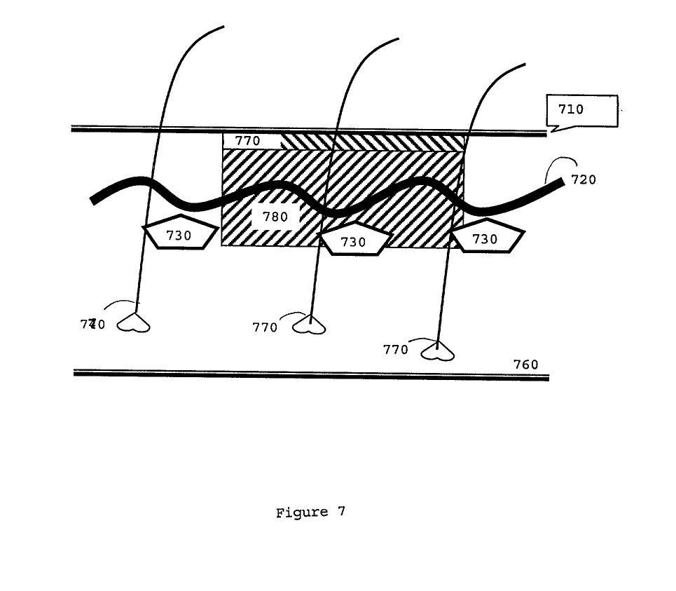

FIG. 7 is a schematic illustration in cross-section of human skin and illustrates the temperature changes at different parts of the skin, the temperature and heat regimen of the present invention to treat acne.

When the heat is applied to the surface of the skin 710, a tightly focused beam, operating for example at 2 W power and a spot size of 200 micrometers for a dwell time of about 0.1 ms can generate peak temperatures of up to a few hundred degrees centigrade in a shallow layer of the epidermis ranging down to about 30 micrometers into the skin. This is layer 770 in FIG. 6.

If sufficient energy is provided, for example about 5 to 10 Joules/cm², then the temperature of deeper layers of the skin, for example, the dermis 780 around and below the papillary dermis 720 as well as sebaceous glands 730 situated below the epidermis, can also be raised to above about 65° C., thus causing modification of the sebaceous gland, reducing sebaceous gland ability to generate sebum which drains into the hair follicles 770. Overproduction of sebum and its accumulation in the hair follicle ducts is the cause of the acne symptoms and its reduction is another advantage of the present invention.

It will be understood by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

What is claimed is:

1. A method for treating skin conditions, including acne, the method comprising the steps:
directing energy capable to the skin for changing the size of pores and follicle openings in the skin.

2. The method of claim 1 wherein the energy directed to the skin does not substantially damage or permanently alter the living cells in the skin.

3. The method of claim 1 wherein the energy directed to the skin changes the size of pores and follicle openings in the skin and modifies sebaceous gland function under the skin.

4. The method of claim 1 further comprising the step of applying the energy to the skin in a continuous wave at about 4 W or less for a duration of about 100 ms or less to a spot on the skin of about 2 micrometers to 2 millimeters in diameter or less.

5. The method of claim 1 further comprising the steps of:
applying the energy to the skin in a continuous wave at about 4 W or less for a duration of about 100 ms or less to a spot on the skin of about 2 micrometers to 2 millimeters in diameter; and
preventing the temperature below the epidermal/dermal junction from rising to more than about 60 degrees centigrade.

6. The method of claim 1 further comprising the steps of:
applying the energy to the skin for a duration of about 100 ms or less to a spot on the skin of about 2 micrometers to about 2 millimeters in diameter; and
preventing the temperature below the epidermal/dermal junction from rising to more than about 60 degrees centigrade.

7. The method of claim 1 further comprising the steps of:
applying the energy to the skin for a duration of about 500 ms or less to a spot on the skin of about 2 micrometers to about 2 millimeters; and
raising the temperature of a sebaceous gland to cause at least some reduction in sebum production by the sebaceous gland.

8. The method of claim 1 further comprising the steps of:
applying the energy to the skin for a duration of about 500 ms or less to a spot on the skin of about 2 micrometers to about 2 millimeters;
raising the temperature of a sebaceous gland to cause at least some reduction in sebum production by the sebaceous gland; and
cooling the surface of the skin at least once after the energy applying step to minimize damage to the surface of the skin.

9. The method of claim 1 further comprising the step of cooling the surface of the skin at least once after the directing energy step.

10. The method of claim 1 wherein the directing energy step modifies the condition of the sebaceous gland; and further comprising the step of cooling the surface at least once after the energy directing step.

11. The method of claim 2 further comprising the step of applying suction to the skin to remove material from the modified pores and follicle openings at least once after the directing energy step.

12. The method of claim 2 further comprising the steps of:
cooling the surface of the skin at least once after the direct energy step; and
applying suction to the skin to remove material from the modified pores and follicle openings at least once after the directing energy step.

13. The method of claim 2 wherein the energy directed to the skin modifies the sebaceous gland to reduce sebum production, further comprising the steps of:
cooling the surface of the skin at least once after the directing step energy; and
applying suction to the skin surface to remove material from the modified pores and follicle openings at least once after the directing energy step.

14. The method of claim 1 wherein the energy is directed to the skin through an intermediate material capable of absorbing the incoming energy, converting it to thermal energy, and transferring the thermal energy to the skin.

15. The method of claim 14 wherein the energy directed to the skin changes the size of pores and follicle openings in the skin.

16. The method of claim 15 wherein the energy directed to the skin modifies the sebaceous gland.

17. The method of claim 15 further comprising the step of applying said energy to the skin for a duration of about 1 ms or less to a spot of about 200 micrometers in diameter or less.

18. The method of claim 17 further comprising the step of preventing the temperature below the epidermal/dermal junction from rising to more than about 60 degrees centigrade.

19. The method of claim 15 further comprising the steps of:
applying said energy to the skin for a duration of about 100 ms or less to a spot of about 2 micrometers to about 2 millimeters in diameter or less; and
preventing the temperature below the sebaceous gland from rising to more than about 55 degrees centigrade.

20. The method of claim 15 further comprising the step of applying said energy to the skin for a duration of about 500 ms or less to a spot of about 2 micrometers to about 2 millimeters in diameter or less so that the temperature of the sebaceous gland is raised to a temperature sufficient to cause irreversible damage to the sebaceous gland.

21. The method of claim 20 further comprising the step of cooling the skin surface at least once after the directing energy step in order to minimize damage to the surface of the skin.

22. The method of claim 15 further comprising the step of cooling the surface of the skin at least once after the directing energy step.

23. The method of claim 22 wherein the directing energy step modifies the sebaceous glands to reduce sebum production.

24. The method of claim 15 further comprising the step of applying suction to the surface of the skin to remove material from the modified pores and follicle openings at least once after the directing energy step.

25. The method of claim 24 further comprising the step of cooling the surface of the skin at least once after the directing energy step.

26. The method of claim 25 wherein the energy directed to the skin causes modification of a sebaceous gland.

27. The method of claim 14 wherein the intermediate material is made of tracing paper.

28. A method for treating skin conditions, including acne, the method comprising the step of directing energy to the skin for modulating physical conditions of the skin by raising the temperature of the top layer of the skin from its surface to about 50 micrometers below the skin surface to between about 45° C. to about 300° C. above the normal skin temperature.

29. The method of claim 28, further comprising the step of preventing the temperature of the skin in the vicinity of the epidermal/dermal junction from rising more than about 30° C.

30. The method of claim 28 wherein the temperature of the top layer of the skin is raised to between about 60° C. to about 200° C. above the normal skin temperature by delivering fluence from about 0.2 Joules/cm² to about 4 Joules/cm² at a spot size of from about 10 micrometers to about 400 micrometers, for a duration of from about 0.1 ms to about 0.5 seconds.

31. The method of claim 28 wherein the temperature of the top layer of the skin to a depth of up to 100 micrometers is raised by between about 20° C. to about 300° C. by delivering fluence ranging from 0.2 Joules/cm² to about 2 Joules/cm² with a dwell time ranging from about 0.1 to about 30 ms so that heat penetration is from about 10 micrometers to about 30 micrometers into the skin.

32. The method of claim 28 wherein the temperature of the skin is raised by between about 20° C. to about 300° C. by delivering fluence ranging from 0.2 Joules/cm² to about 2 Joules/cm² with dwell time ranging from about 0.1 to about 1 ms, raising a beam spot from about 50 micrometers to about 600 micrometers in size scanned over an area of from about 0.3 cm² to 16 cm², so that heat penetration is from about 10 micrometers to 30 micrometers into the skin.

33. The method of claim 28 wherein the temperature of the top layer of the skin from its surface to about 300 micrometers below the skin surface is raised from between about 60° C. to about 100° C. above its normal temperature by delivering energy to a continuous wave form fluence from about 2 Joules/cm² to about 4 Joules/cm² at a spot size of from about 100 micrometers to about 400 micrometers for a duration of from about 0.1 ms to about 0.5 seconds.

34. A device for treating skin conditions, the device comprising an energy source capable of modulating physical conditions of the skin to open pores in the skin.

35. The device of claim 34 wherein the energy source heats a portion of the skin.

36. The device of claim 34 wherein the energy source heats a portion of the skin displacing a portion of the skin with respect to another unheated portion.

37. The device of claim 34 further comprising a vacuum source for applying suction to the surface of the skin to enhance removal of unwanted substances from the skin.

38. The device of claim 34 wherein the energy source modulates the physical conditions of the skin by mechanically displacing a portion of the skin to open pores of the skin.

39. A device for treating skin conditions, the device comprising:
an energy source for directing energy to a skin surface to be treated; and
a substance capable of safely contacting the skin interposed between said energy source and the skin, said substance absorbing energy from the energy source and transmitting to the skin.

40. The device of claim 39 wherein the energy transmitted to the skin by the substance is thermal energy.

41. The device of claim 39 wherein said energy source generates electromagnetic energy.

42. The device of claim 39 wherein said energy source is a laser.

43. The device of claim 39 wherein said energy source is a laser and said substance absorbs the energy from the laser.

44. A device for treating skin conditions, the device comprising:
a laser energy source; and
a substance capable of safely contacting the skin interposed between the laser and the skin for transmitting thermal energy to the skin in response to laser energy from the laser energy source.

45. The device of claim 44 further comprising a focusing element capable of focusing the laser energy from the laser energy source into a small spatial location on the substance.

46. The device of claim 45 further comprising reflective or refractive elements capable of moving the focused laser energy in space so that the small spatial location on the substance is moved along a predetermined larger area of the substance.

47. The device of claim 46 further comprising:
a controller capable of activating the energy source; and
a controller for triggering and controlling the reflective or refractive elements to move the focused laser energy in space.

48. A device for treating skin conditions, the device comprising:
an electromagnetic energy source directing electromagnetic energy to the skin; and
a substance interposed between the energy source and the skin capable of safely contacting the skin, said substance transmitting part of the electromagnetic energy to the skin and absorbing part of the electromagnetic energy.

49. The device of claim 48 wherein the electromagnetic energy absorbed by the substance is converted to heat and conducted to the skin.

50. A device for treating skin conditions, the device comprising:
an electromagnetic energy source;
a substance interposed between the energy source and skin capable of safely contacting the skin;
the substance transmitting part of the electromagnetic energy from the source to the skin while absorbing the remaining part;
the absorbed electromagnetic energy being converted to heat and conducted to the skin; and
the transmitted electromagnetic energy impinging on the skin converted to heat at the upper layers of the skin.

51. The device of claim 50 wherein the transmitted electromagnetic energy impinging on the skin is converted to heat at tissue layers below the skin surface, whereby a heating effect occurs at the skin surface and at tissue layers below the skin surface.

52. The device of claim 51 wherein said substance comprises a surface which is covered by absorbing material from about 2% of its surface to about 50% of its surface, whereby electromagnetic energy travels through the part of the surface not covered by absorbing material.

53. The device of claim 50 further comprising a vacuum source for generating a vacuum to remove debris and unwanted substances from the skin surface to help further open hair follicle openings and pores in the skin.

54. A device for treating skin conditions, the device comprising:
an energy source; and
a focusing element to focus energy from the energy source on a location on the skin thereby creating displacement of one part of the skin with respect to an adjacent part of the skin.

55. A device for treating skin conditions, the device comprising:
an energy source; and
a conduit capable of transferring energy from the energy source to the skin thereby changing the size of pores and follicle openings in the skin.

56. The device of claim 55 further comprising an element for redirecting energy from the energy source from one location on the skin to another location on the skin.

57. The device of claim 56 wherein the element for redirecting energy causes a larger area to be blanketed with thermal energy, elevating the temperature of the larger area.

58. The device of claim 57 whereby the temperature of the layer is elevated sufficiently to cause death of bacteria in upper layers of the skin.

59. The device of claim 56 further comprising a cooling substance capable of removing thermal energy from the skin at a desired time.

60. A device for treating skin conditions, the device comprising:
an electromagnetic energy source; and
a substance interposed between the energy source and skin capable of safely contacting the skin, the substance substantially absorbing most of the energy from the energy source and substantially transmitting mostly thermal energy to the skin.

61. The device of claim 60 wherein said energy source emits microwave energy.

62. The device of claim 61 wherein said microwave energy is focused on a small location within the skin surface so as to effect a differential heating of one part of the skin while maintaining the other part of the skin at a lower temperature.

63. The device of claim 61 wherein said microwave energy is focused on a small location within the skin surface so as to effect a differential heating of one part of the skin while maintaining the other part of the skin at a lower temperature so that a portion of the skin in the vicinity of a hair follicle is displaced with respect to an adjacent region of the skin, the differential displacements of skin regions opening the follicles and releasing substances trapped underneath the skin surface.

64. The device of claim 63 wherein said microwave energy reduces sebum production in the sebaceous gland and further comprising a source for a substance capable of removing energy from the skin surface.

65. A device for treating skin conditions, the device comprising:
an RF energy source; and
a focusing element for focusing the RF energy from the source to a small location within the skin surface to change the size of pores and follicle openings in the skin.

66. The device of claim 65 wherein the application of RF energy to one part of the skin causes opening of the follicles and releasing substances trapped underneath the skin surface.

67. A device for treating skin conditions, the device comprising:
an electrical energy source; and
a focusing element for focusing the electrical energy on a small location within the skin surface to change the size of pores and follicle openings in the skin, opening the follicles or pores, and releasing substances trapped underneath the skin surface.

68. A device for treating skin conditions, the device comprising:
a plasma energy source; and
a focusing element for focusing the plasma energy from the source on a small location within the skin surface to change the size of pores and follicle openings in the skin, opening the follicles or pores, and releasing substances trapped underneath the skin surface.

69. A device for treating skin conditions, the device comprising:
a chemical energy source; and
the chemical energy from the source being focused on a small location within the skin surface so as to change the size of pores and follicle openings in the skin, opening the follicles or pores, and releasing substances trapped underneath the skin surface.

70. A device for treating skin conditions, the device comprising:
an ultrasonic energy source; and
a focusing element for focusing the ultrasound energy to a small location within the skin surface to change the size of pores and follicle openings in the skin, opening the follicles or pores, and releasing substances trapped underneath the skin surface.

71. A device for treating skin conditions, the device comprising:
a mechanical energy source; and
a focusing element for focusing the mechanical energy on a small location within the skin surface to change the size of pores and follicle openings in the skin, opening the follicles or pores, and releasing substances trapped underneath the skin surface.

72. A method for treating skin conditions, the method comprising the step of directing energy to the skin to be treated to change the size of pores and follicle openings in the skin, opening the follicles or pores, and releasing substances trapped underneath the skin surface.

73. The method of claim 72 further comprising the step of focusing the energy on the skin to a spot between about 1 micrometer and about 2 mm in size.

74. The method of claim 72 further comprising the steps of:
focusing the energy on the skin to a spot between 1 micrometer and 2 mm in size onto the skin; and
moving the focused energy spot on the skin surface so that a larger portion of the skin surface is covered.

75. The method of claim 72, further comprising the steps of:
focusing the energy to a spot between about 1 micrometer and about 2 mm in size onto an intermediate material capable of absorbing said energy and transmitting at least some of the energy to a target region of the skin in contact with the intermediate material to create differential heating and differential displacement of one part of the skin with respect to an adjacent part of the skin; and
moving the focused energy spot on the intermediate material so that a larger portion of the intermediate material surface is covered.

76. The method of claim 72 for treating skin conditions, the method comprising:
focusing the energy to a spot between 1 micrometer and 2 mm in size onto an intermediate material capable of absorbing said energy and transmitting at least some of the energy to a target region of the skin in contact with the intermediate material to create differential heating and differential displacement of one part of the skin with respect to an adjacent part of the skin;
moving the focused energy spot on the intermediate material so that a larger portion of the intermediate material surface is covered; and
applying a substance capable of removing energy from the intermediate material before, after, or during the direction of the energy to the intermediate material.

77. A method for treating skin conditions, the method comprising directing energy to the skin for changing the size of pores and follicle openings in the skin without substantially damaging live tissue and live cells.

78. The method of claim 77 wherein the damage on live tissue and live cells being avoided is vaporizing or ablating live tissue.

79. The method of claim 77 wherein the damage on live tissue and live cells being avoided is irreversible changes to live tissue and living cells.

80. The method of claim 77 wherein the damage on live tissue and live cells being avoided is coagulating or denaturing live tissue and living cells.

81. The method of claim 77 wherein the damage on live tissue and live cells is avoided by not raising the temperature of the tissue at and below the epidermal/dermal junction to more than about 45° C.

82. The method of claim 77 wherein the damage on live tissue and live cells is avoided by not raising the temperature of the tissue at and below the bottom of the sebaceous gland to more than about 65° C.

83. A method for treating skin conditions, including acne, the method comprising the step of modifying the skin top layer to a depth sufficient to remove blockage of follicle openings to allow removal of unhealthy substances in the follicle openings.

84. The method of claim 83 further comprising the step of directing external energy to the top layer of the skin to cause removal of blockage of follicle openings.

85. The method of claim 83 wherein the external energy is electromagnetic energy.

86. The method of claim 83 wherein the external energy is thermal energy.

87. The method of claim 83 wherein the external energy is ultrasound energy.

88. The method of claim 83 wherein the external energy is microwave energy.

89. The method of claim 83 wherein the external energy is RF energy.

90. The method of claim 83 wherein the external energy is laser energy.

91. The method of claim 83 wherein the external energy is solid state diode energy.

92. The method of claim 83, further comprising the steps of:
the external energy being absorbed by an intermediate energy absorbing barrier;
converting at least some of the external energy into heat, the intermediate barrier conducting the heat to the skin.

93. The method of claim 92, further comprising the step of applying a coolant to the intermediate barrier before, during, or after energy application.

94. The method of claim 92, further comprising the step of applying alcohol or other sterilizing agents to the skin surface before, during, or after energy application.

95. The method of claim 92, further comprising the step of applying vacuum suction to the skin before, during, or after energy application.

96. The method of claim 92, further comprising the step of applying antibiotics to the skin before, during, or after energy application.

97. The method of claim 92, further comprising the step of applying drug agents, oxygen, or nutrients to the skin before, during, or after energy application.
